(19) United States
(12) Patent Application Publication
(10) Pub. No.: US 2010/0161267 A1
(43) Pub. Date: Jun. 24, 2010

(54) Device and Method for Automatic Calibration Verification of an Analyzer

(75) Inventors: Arnold Bartel, Graz (AT); Bernhard Engel, Murau (AT)

Correspondence Address: ROCHE DIAGNOSTICS OPERATIONS INC., 9115 Hague Road, Indianapolis, IN, 46250-0457, US

(73) Assignee: ROCHE DIAGNOSTICS OPERATIONS, INC., Indianapolis, IN (US)

(21) Appl. No.: 12/638,139

(22) Filed: Dec. 15, 2009

Related U.S. Application Data
(60) Provisional application No. 61/139,173, filed on Dec. 19, 2008.

(51) Int. Cl. G06F 19/00; G01N 33/48

(52) U.S. Cl. 702/100

(57) ABSTRACT

A method and a device for carrying out calibration verification (CV process) of an analyzer for determining different measurement parameters in body fluids is provided. The device according to the invention comprises the following components: a cartridge with a plurality of containers, which can be connected to an intake system of an analyzer, each of which contains a measurement fluid with known measurement parameter values; a storage unit assigned to the cartridge for storing the measurement parameter values of the measurement fluids and data relevant for the CV process of the analyzer to be verified; a docking or intake area at or in the analyzer for receiving the cartridge with a data path for reading the storage unit; an evaluation unit typically housed in the analyzer for evaluating the values measured by the analyzer; and an output unit for printing an evaluation protocol.

### BACKGROUND OF THE INVENTION

The invention relates to a device and method for carrying out a verification of the calibration (calibration verification) of an analyzer used to determine different measuring parameters in body fluids.

In medical diagnostics analyzers are used for decentralized determination of different parameters and groups of parameters in body fluids, especially in whole blood, serum, plasma, urine and dialysate samples. Measurements are carried out by means of optical, chemical, biochemical and electrochemical sensors, and spectroscopic methods.

The sensors measure concentrations, activities, partial pressures and other characteristics of chemical, biochemical and biological substances present in the samples.

A group of parameters or a parameter panel is a certain combination of individual parameters, such as concentrations or activities of metabolites (glucose, lactate, urea, creatinine, etc.), of electrolytes (K, Na, Ca, etc.), partial pressures of blood gases (O₂, CO₂), pH, hematocrite value, hemoglobin values (tHb, SO₂, etc.), and all quantities and values derived from these.

### Calibration

As a rule the measuring system or the individual sensors have to be calibrated at certain predetermined times. Calibration, which usually is carried out automatically by the analyzer, is a method for determining the characteristic curves of the sensors. During calibration the sensors are brought into contact with calibrating media, which contain one or more analytes in various known concentrations. To determine the characteristic curves of the individual sensors, essentially the signals of the sensors are obtained from a series of aqueous calibrating media containing various known concentrations of the relevant analytes distributed over the expected range of the measured variable, thus providing one or more calibrating values distributed over the measurement range. With the use of these calibrating values the characteristic curve is determined.

### Quality Control

Besides calibration and independently thereof, quality control measures (QC measurements) are carried out in the analyzers mentioned. In general, control fluids (QC fluids) are used, which are independent of the calibration means in the analyzer and which have known parameter values (target values), usually for a group of parameters. These quality control fluids, which usually are contained in glass ampoules, are introduced into the measuring system of the analyzer and measured values of the parameters activated in the analyzer are obtained (actual values). Subsequently each (activated) measurement parameter is checked for whether the actually measured values lie within a certain predetermined tolerance region around the target values. Evaluation is optionally carried out either by the evaluation unit of the analyzer or by the user. QC measurement is usually done with only one QC fluid or one QC ampoule per control measurement.

The frequency of QC measurements and the number of QC fluids to be used—each fluid at various values (“levels”) of the parameter to be determined—are prescribed by national standards. RiliBÄK (Richtlinie der Bundesärztekammer, Germany) demands one QC measurement per analyzer batch or working shift and a change of level of the measured parameter between QC measurements. CLIA (Clinical Laboratory Improvement Amendments, U.S.A.) also requires a QC measurement following each eight-hour measuring shift, which is to be carried out by means of at least two control fluids, the first containing high levels and the second low levels of the parameters to be measured. The exact value of a measuring parameter relative to the expected measurement range of the parameter is not prescribed. In general a medium level will correspond to the normal physiological values. Analogously, low and high levels correspond to physiologically decreased or increased values. As a rule the levels mentioned will cover only part of the possible measuring range of the individual parameters.

In quality control (depending on the valid regulations) daily (or at least frequent) manual or automatic measuring of QC fluids and comparing of the measured values with the target values of the control samples is state of the art. A well-known system performing this process automatically is cobas b 221 made by Roche Diagnostics.

### Calibration Verification

In addition to frequent QC measurements the regulations of some countries require calibration verification of such systems by means of a check which covers the complete measuring range. Calibration verification will henceforth be abbreviated “CV”. The method used in carrying out CV will be referred to as the “CV process”.

For the purposes of CV, measured values for measurement parameters are required not only in the normal measuring range (usually around the center of the measurement range) but also at both ends (low-end, high-end) of the total measurement range. It is especially desirable for CV to obtain for each measurement parameter not only a low, a medium and a high parameter value but also a very low value at the lower limit of the measurement range and a very high value at the upper limit of the measurement range.

In a measuring system for determination of the pH-value of whole blood, CV for example comprises the repeated measurement of five levels, typically with three independent measurings at each level (see table in FIG. 2). In this variant CV of the measuring system for pH determination requires measurement of a total of 15 fluids provided in ampoules.

In practice, deviations from ideal linearity between actual and target values may occur due to a variety of causes. Sufficient linearity for a given measurement parameter is given if over the total range the measured value (actual value) of the measurement parameter lies within a predetermined tolerance interval around the target value of the measurement parameter. A measure of deviation of the individual measured points from the ideal line A (see FIG. 3, dashed line) and thus a measure for the deviation of measured values from target values is computed from the values distributed over the total measurement range.

In the context of a CV process it is thus determined whether the measured values or quantities derived therefrom lie within a predetermined tolerance region.

For this purpose the following criteria or statistical variables may be used either independently or in combination.
the absolute, relative or percentage deviation of the mean values of the measured values per level from the target values;
generally known statistical measures of dispersion;
the parameters of a regression line B (see FIG. 3, full line);
the deviation of the regression line B from the ideal line A;
the shift or rotation of regression line B relative to the ideal line A;
deviations of data points from the regression line B;
the coefficient of multiple determination;
statistical evaluation of the position of measured data points relative to the ideal line A (e.g., how many measured values lie above/below the target value, what is the distance of the measured values above/below the target value);
confidence intervals (e.g., of the mean value of measured values or of the regression parameters);
graphical representation.

CV for other measurement systems integrated in the analyzer, such as systems for determining concentrations or activities of metabolites (glucose, lactate, urea, creatinine, etc.), of electrolytes (K, Na, Ca, etc.), for determining the partial pressures of blood gases (O₂, CO₂), for measuring of hemoglobin values (tHb, SO₂, etc.), of the hematocrite, etc., is carried out in complete analogy to the example of the pH measurement system.

CV usually is required at comparatively large intervals (usually biannual periods) or when the measuring system has been subject to changes (e.g., after an exchange of sensors).

CLIA for example prescribes a CV at least once every six months, with the measurement samples covering the complete measuring range and comprising at least very low (or zero values), medium and very high measurement parameter values near the upper end of the measurement range.

CV thus is a process which, in addition to and independent of calibration and QC, checks the reliability of the analytic system over the complete parameter panel and especially over the complete measuring range of the individual measurement parameters. This can be carried out by comparison of measured and target values of control fluids, or by the additional determination of evaluation parameters based on a plurality of measured values (dispersion of multiple measurements of one and the same concentration, linearity (e.g., deviation from an ideal line through all concentrations of a measurement parameter)).

So far measurements of the control fluids in the CV process have been carried out manually by the user in accordance with specifications of the control fluid manufacturer or with the operations manuals of the manufacturer of the apparatus. Evaluation of the results, interpretation and presentation or documentation of the results is also done by the user.

In known applications a measurement series for CV is carried out by measuring fluids provided in ampoules and can be quite time-consuming, since—depending on the parameter panel—typically five to seven different fluids are used, each requiring three measurings.

Depending on the parameter panel the user thus must manually perform 15 to 21 measurements with the analyzer using CV media which come in ampoules; he has to select the ampoules according to a certain plan, must open them, bring them in contact with the intake device of the analyzer, start the measuring process, read the results from a display or a printout of the analyzer and enter them, usually manually, into a separate computer, evaluation of the results being performed by a separate tool.

Carrying out such measurement series is thus very time-consuming and work-intensive. Each individual measurement typically takes a few minutes.

Evaluation by means of a separate tool moreover is difficult and error-prone, considering the fact that transfer of the measured data from display or printout, and reading and correct assignment of the target values from the manufacturer's specifications has to be done manually by the user.

### SUMMARY OF THE INVENTION

It is against the above background that the present invention provides certain unobvious advantages and advancements over the prior art. In particular, the inventors have recognized a need for improvements in devices and methods for automatic calibration verification of an analyzer.

Although the present invention is not limited to specific advantages or functionality, it is noted that the present invention simplifies and facilitates measurements of control media and their evaluation for the calibration verification process (CV process) and to exclude sources of error as far as possible.

In accordance with one embodiment of the invention, a method for carrying out a verification of the calibration (calibration verification, CV) of a measurement system of an analyzer for determining different measurement parameters in body fluids is provided, the method comprising the steps of:
inserting into or docking onto the analyzer a cartridge containing a plurality of identical sets of at least five different measurement fluids with known different measurement parameter values, which are distributed over the total measurement range of the measurement parameters which can be measured by the measurement system, in particular including measurement parameter values at the upper and lower limits of the measurement ranges,
reading in the measurement parameter values stored in a storage unit assigned to the cartridge, and other data and information relevant for the CV process of the analyzer to be verified,
selecting measurement fluids to be measured according to the data stored in the storage unit,
feeding the measurement fluids into the measurement system by the analyzer and measuring them by the analyzer,
evaluating the measurement results automatically by an evaluation unit assigned to the analyzer, taking into account the stored data, and
outputting an evaluation protocol on an output device.

Evaluation of the measured results is typically done by the methods mentioned above, e.g., by computing a measure for the deviation of the measured points from an ideal line A and/or other methods described in this context.

According to an embodiment of the invention, the user is supplied with all the fluids required in the CV process (contained for instance in ampoules) together with a data chip containing all necessary information and instructions for the CV process, in the form of a closed cartridge (CV box), which is specifically designed for CV measurements and is to be inserted into the analyzer, the latter carrying out the measurement process automatically—using the information contained in the data chip—and transferring the measurement results automatically to an evaluation unit where the parameter-specific quantities of the CV process are computed and displayed.

For each measurement parameter and level three measured values are typically obtained, requiring three identical sets of different measurement fluids.

The invention further provides that the selection of measurement fluids may be limited to only those measurement parameters activated in the analyzer. Thus only control measurements corresponding to the actual measurement parameter configuration of the analyzer are carried out automatically.

Guided by the results of the CV process the user will take suitable measures, possibly aided by a list of possible activities on the display of the analyzer.

In further enhancement of the invention the analyzer interprets the results of the evaluation protocol and automatically initiates suitable or necessary measures for maintaining operation according to specifications or for restricted operation. Individual measurement parameters or groups of parameters of the parameter panel may for instance be disabled automatically, while the analyzer will stay operational for the remaining measurement parameters. Disabling may mean that a measurement parameter or group of measurement parameters is completely unavailable, or there may be partial disabling by restricting the available measurement range of the parameter or group of parameters. For instance, measuring of metabolites (glucose, lactate, urea, creatinine, etc.) may be disabled and the analyzer used only for blood gas measurement (O₂, CO₂) and pH measurement, until the cause of the disturbance has been eliminated.

According to another embodiment of the invention, a device for carrying out the CV of an analyzer for the determination of different parameters of body fluids is provided and comprises the following elements:
a cartridge with a plurality of sets of containers, which can be connected to an intake system of an analyzer, each set of containers comprising at least five different measurement fluids with known different measurement parameter values, which are distributed over the total measurement range of the measurement parameters which can be measured by the measurement system, in particular including measurement parameter values at the upper and lower ends of the measurement ranges;
a storage unit assigned to the cartridge for storing the measurement parameter values of the measurement fluids and data and information relevant for the CV process of the analyzer to be verified;
a docking or intake area at or in the analyzer for receiving the cartridge with a data path for reading the storage unit;
an evaluation unit for evaluating the values measured by the analyzer; and
an output unit for printing an evaluation protocol.

In a variant of the invention the analyzer and the CV evaluation unit may be configured as two separate units, with automated communication (data transfer) between analyzer and evaluation unit. In another variant the evaluation unit and/or the output unit are modules of the analyzer or are identical with corresponding devices within the analyzer.

The cartridge typically has at least three sets of containers with different measurement fluids.

The storage unit may typically be a data chip attached to the cartridge. Moreover, the storage unit may be configured as a storage medium which is enclosed in the package of the cartridge and must be inserted or introduced into the analyzer by the user.

These and other features and advantages of the present invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following detailed description of the embodiments of the present invention can be best understood when read in conjunction with the following drawings, where like structure is indicated with like reference numerals and in which:

FIG. 1 is a schematic drawing of an analyzer with a device according to the invention for carrying out a CV process;

FIG. 2 is a table showing the measurement parameter values for three QC levels and six CV levels within the limits of the corresponding measurement ranges;

FIG. 3 is a chart of the comparison between measured values and target values in the case of a pH-measurement unit;

FIG. 4 is a chart of the results of a CV measurement; and

FIG. 5 is a variant of the chart of FIG. 4.

Skilled artisans appreciate that elements in the figures are illustrated for simplicity and clarity and have not necessarily been drawn to scale. For example, the dimensions of some of the elements in the figures may be exaggerated relative to other elements to help improve understanding of the embodiment(s) of the present invention.

### DETAILED DESCRIPTION OF THE INVENTION

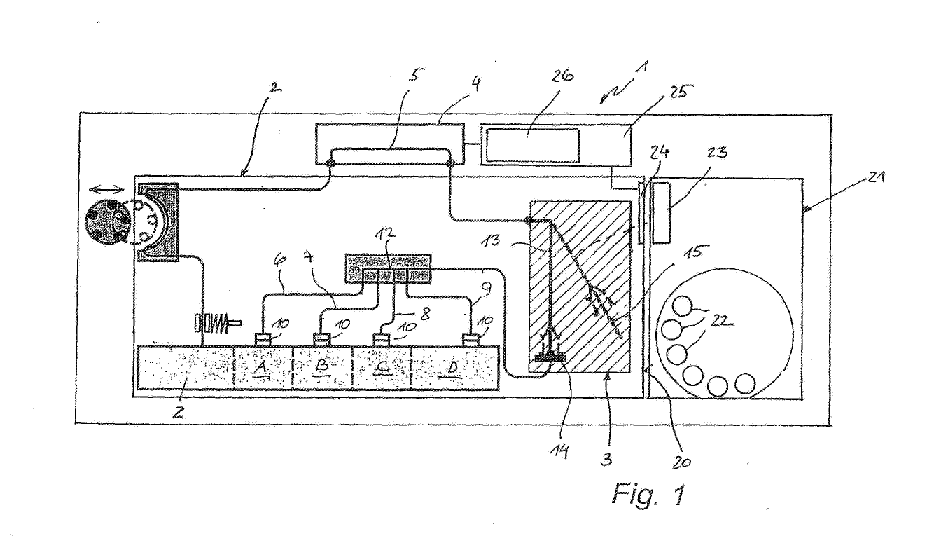

The analyzer 1 for medical sample fluids, for instance blood samples, schematically shown in FIG. 1, has a reagent cartridge 2 which can be exchangeably inserted into the analyzer 1. The cartridge 2 contains a number of bags A to D for operational fluids, and a waste container Z, the bags containing functional fluids, such as calibrating fluids, quality control media, flushing and rinsing fluids and disinfectants, which can selectively be fed into an intake system 3 and further on into a measurement chamber 5, which is for instance situated in a sensor cartridge 4. The intake system 3 of the analyzer 1 has a tiltable input element 13 (for instance a hollow needle), which in a rest position connects to a docking element 14 for supplying calibrating and rinsing media, and which can take in sample fluids when tilted away from the rest position to a position 15.

Each bag of operational fluid A to D has a multiway valve 10 directly at the fitting of its connecting line 6, 7, 8, 9, which valve 10 is controlled by the analyzer. All connecting lines 6, 7, 8, 9 of the bags A to D departing from the multiway valves 10 open into a commen collector line 12, which connects to the docking element 14 of the sample input system 3.

The analyzer 1 has a docking or receiving area 20 for a cartridge 21 (CV box), with the cartridge 21 holding a plurality of containers 22 (sealed ampoules or cuvettes), each containing a measurement fluid with known measurement parameter values, which containers can be brought into contact with the input system 3 of the analyzer. Possible designs of devices for the opening and removing of ampoules and for moving them towards the intake system of the analyzer are for instance described in U.S. Pat. No. 6,099,510 A or U.S. Pat. No. 5,628,353.

The cartridge 21 is furnished with a storage unit 23 (data chip) for storing the measurement parameter values of the measurement fluids and the information relevant for the CV process of the analyzer to be verified. In the docking or receiving area 20 at or in the analyzer a data path 24 for reading out the storage unit 23 is provided. Data transfer may occur via a connector socket, by wireless or by optical means. Furthermore there is provided an evaluation unit 25 for evaluating the CV measurements obtained by the analyzer, the evaluation unit being connected to an output unit 26 for printing an evaluation protocol.

The storage unit 23 integrated in the CV box 21 will typically contain not only information relating to the target values of the measurement parameters assigned to the individual fluids, but also information concerning the execution of the CV process itself and the evaluation of results. The storage unit may for instance provide information on how to obtain measurement results for the individual measurement parameters, and how to evaluate and present these results.

A CV process may be carried out for all measurement parameters or for a subset of parameters. This will for instance depend on which measurement parameters are activated in the analyzer. The apparatus may for instance accept different sensor cartridges 4 for different measurement parameters or groups of measurement parameters, for which different CV processes can be provided or activated. There may furthermore be different configurations of the apparatus. The CV box is typically suitable for varying configurations or equipment (e.g., different sensor cartridges). A first variant of the apparatus may for instance contain an oximeter for the photometric determination of hemoglobin parameters in addition to sensors for determining blood gases, electrolytes and metabolites, while a second variant does not contain such an oximeter. In this case the system can determine which measurements are to be performed, evaluated and presented for the CV process, in accordance with the data provided by the chip and the analyzer—without intervention by the user.

The closed CV box 21 may in one variant be equipped with a plurality of containers or ampoules 22 (for instance, ampoules for seven different fluids arranged in a circle) with combinations of measurement parameter concentrations. In accordance with the parameter panel available or selected in the analyzer, the fluids required for covering the desired ranges of the individual measurement parameter values may be selected, measured and the results evaluated.

In accordance with the present invention, the user need not manually carry out the CV measurements. This reduces the work load and saves time, especially since the analyzer evaluates and presents the measurement results automatically. User errors due to bad handling or erroneous selection of ampoules 22 are avoided. A further advantage lies in the automated reading of the limits of measurement ranges of individual parameters from the data chip 23 and in their automated use in evaluation, thus excluding any confounding or mixing of data.

A graphical chart is produced as evaluation protocol or in addition to a written presentation of the evaluation protocol, in which the measured values of individual parameters (actual values) or statistical variables derived therefrom are plotted against the stored target values, taking into account admissible limits.

The table below presents an example of a CV process according to the invention for an analyzer as shown in FIG. 1.

| | |
|---|---|
| 1 | Initiating the CV process by selecting the appropriate menu entry on the analyzer 1. |
| 2 | Inserting into or docking onto the analyzer the CV box 21 of the invention. |
| 3 | The analyzer reads information relevant for the CV process from the data chip 23, and determines |
| | the ampoules 22 to be measured in accordance with the parameter panel available or selected on |
| | the analyzer. |
| 4 | The analyzer opens the CV box 21. |
| 5 | The analyzer selects an ampoule 22, opens it, lifts it and moves it automatically towards the intake |
| | system 3 of the analyzer 1. |
| 6 | The fluid is fed into the measuring chamber 5 and measured. |
| 7 | The individual measured values are compared with the target values of the data chip 23. |
| 8 | The individual measured values and the evaluation are stored. |
| 9 | Other ampoules 22 are measured: the steps 5 to 8 are repeated. |
| 10 | At least one of the following parameter-specific quantities is computed in the evaluation unit 25 from |
| | the individual measurements of step 6: |
| | mean value of individual measurements at one concentration |
| | standard deviation of the individual measurements at one concentration |
| | linearity (e.g., deviation from an ideal line through all concentrations of a measurement parameter) |
| 11 | An evaluation protocol is produced and presented on an output unit 26 (display, printout and/or |
| | stored file). |
| 12 | (possibly) Automated follow-up measures such as disabling of measurement parameters, shutdown |
| | of a consumable medium, etc.. |

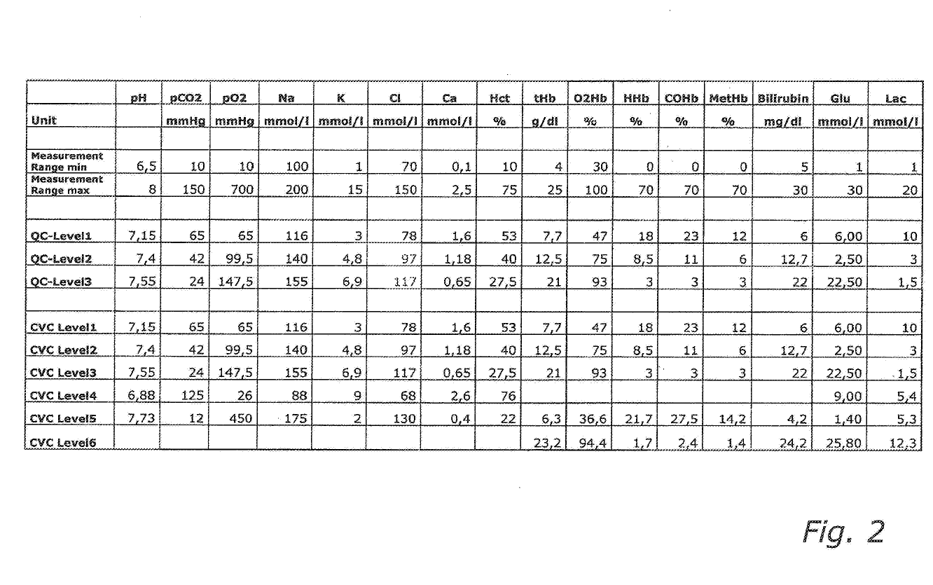

The table presented in FIG. 2 contains typical measurement parameters and their measurement ranges for blood gas analyzers. It also shows the corresponding measurement parameter values for 5 and/or 6 fluids with different measurement parameter values distributed over the total measurement range (here labelled CVC Level1 to Level6). The fluids labelled Level1 to Level3 may also be used for daily QC measurements. The fluids labelled Level4, Level5 and Level6 have—for each of the measurement parameters shown—very high and very low measurement parameter values covering the upper and lower limits of the measurement range of the individual parameters. In the present case a sixth fluid (CVC Level6) is additionally used for a special group of measurement parameters (in this instance for hemoglobin derivates, etc.). In contrast to the QC solutions, the CV solutions cover most of the measurement range.

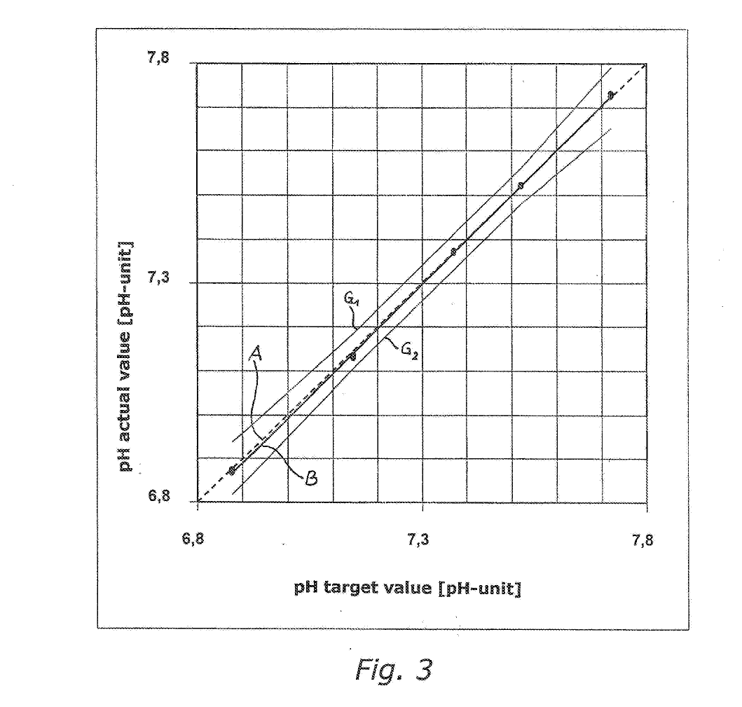

FIG. 3 is a graphic presentation of the results of a CV measurement. Fifteen pairs of values are plotted (see table below), the data points overlapping to a great degree due to very similar measurement results. The measured values were obtained from 15 measurements of 15 fluid ampoules with five different pH levels. Three measurements were taken at each of the five pH levels. Variance of the measured data at each level was so small that the differences cannot be discerned in the plot. The dashed line A represents the ideal case of measured values fully coinciding with target values. The full line B is the regression line computed from the 15 value pairs. The area between the upper and lower boundary curve G₁ and G₂ is the tolerance region for measured values.

The table below contains the measured pH values from 15 measurements, with three measurements taken at each of five pH levels (measurement 1, measurement 2, measurement 3). Also shown are the pH target values (Xₛ) at the five pH levels, and the tolerable deviations (LAO upwards or downwards at these levels. Xₘ are the means, SD the standard deviations, CV coefficients of variation as percentages, Xₘ−Xₛ deviations of the means from the respective target values, ((Xₘ−Xₛ)/Xₛ)×100 the relative deviations as percentages.

| | | | | | | | | | Deviation of mean | |
|---|---|---|---|---|---|---|---|---|---|---|
| | | | Measured values | | | | | | from target value | |
| | Target values | | Measurem. | Measurem. | Measurem. | | | | | (Xₘ − Xₛ)/ |
| | Xₛ | ΔXₜₒₗ | 1 | 2 | 3 | Xₘ | SD | CV % | Xₘ − Xₛ | Xₘ × 100 |
| Level 4 | 6.877 | 0.060 | 6.869 | 6.870 | 6.873 | 6.871 | 0.0020 | 0.029 | −0.006 | −0.094 |
| Level 1 | 7.146 | 0.040 | 7.130 | 7.131 | 7.134 | 7.132 | 0.0023 | 0.032 | −0.014 | −0.201 |
| Level 2 | 7.372 | 0.040 | 7.370 | 7.372 | 7.370 | 7.371 | 0.0007 | 0.010 | −0.001 | −0.017 |
| Level 3 | 7.521 | 0.040 | 7.521 | 7.522 | 7.521 | 7.521 | 0.0002 | 0.003 | −0.000 | 0.000 |
| Level 5 | 7.722 | 0.070 | 7.729 | 7.725 | 7.727 | 7.727 | 0.0017 | 0.022 | −0.005 | 0.066 |

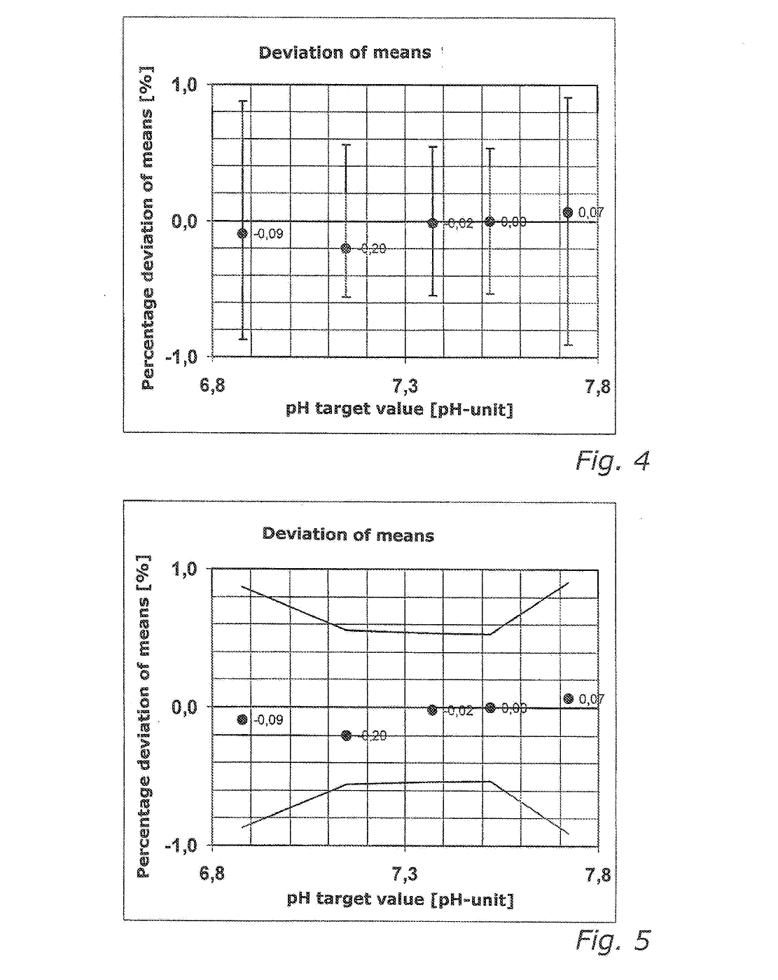

The evaluation of a CV measurement includes at each of the five levels for instance the computation of the mean (Xₘ), the standard deviation (SD), the coefficient of variation (CV), the deviation (difference) of the mean from the target value (Xₘ−Xₛ), a statement concerning linearity and a graphical plot of the data as shown in FIG. 4 or 5.

FIGS. 4 and 5 show possible graphical representations of the results of a CV measurement. Percentage deviations of the means from the target values (zero line) are plotted. In FIG. 4 the bars show the tolerance intervals for deviations of measured values at the individual pH levels. In analogy, the upper and lower broken lines in FIG. 5 represent the boundaries of the tolerance region for the measured values.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in is a change in the basic function of the subject matter at issue.

Having described the invention in detail and by reference to specific embodiments thereof, it will be apparent that modifications and variations are possible without departing from the scope of the invention defined in the appended claims. More specifically, although some aspects of the present invention are identified herein as preferred or particularly advantageous, it is contemplated that the present invention is not necessarily limited to these preferred aspects of the invention.

What is claimed is:

1. A method for carrying out a verification of the calibration (calibration verification, CV) of a measurement system of an analyzer for determining different measurement parameters in body fluids, comprising the steps of:
inserting into or docking onto the analyzer a cartridge containing a plurality of identical sets of at least five different measurement fluids with known different measurement parameter values, which are distributed over the total measurement range of the measurement parameters which can be measured by the measurement system, in particular including measurement parameter values at the upper and lower limits of the measurement ranges,
reading in the measurement parameter values stored in a storage unit assigned to the cartridge, and other data and information relevant for the CV process of the analyzer to be verified,
selecting measurement fluids to be measured according to the data stored in the storage unit,
feeding the measurement fluids into the measurement system by the analyzer and measuring them by the analyzer,
automatic evaluation of the measurement results by an evaluation unit assigned to the analyzer, taking into account the stored data, and
output of an evaluation protocol on an output device.

2. The method according to claim 1, wherein at least three identical sets of at least five different measurement fluids each are used.

3. The method according to claim 1, wherein the selection of the measurement fluids to be measured is restricted to the measurement parameters activated in the analyzer.

4. The method according to claim 1, wherein the evaluation protocol comprises a written and/or a graphical presentation, in which the measured values of the individual measurement parameters (actual values) and/or statistical variables derived therefrom are compared with stored target values, taking into account permissible limits.

5. The method according to claim 1, wherein the analyzer, depending on the results of the evaluation protocol, automatically initiates suitable or necessary measures for maintaining the measuring operation.

6. The method according to claim 5, wherein individual measurement parameters or groups of measurement parameters of the parameter panel are disabled.

7. A device for carrying out a calibration verification process (CV process) on an analyzer for determining different measurement parameters in body fluids, which comprises
a cartridge with a plurality of sets of containers, which can be connected to an intake system of an analyzer, each set of containers comprising at least five different measurement fluids with known different measurement parameter values, which are distributed over the total measurement range of the measurement parameters which can be measured by the measurement system, in particular including measurement parameter values at the upper and lower ends of the measurement ranges;
a storage unit assigned to the cartridge for storing the measurement parameter values of the measurement fluids and data and information relevant for the CV process of the analyzer to be verified;
a docking or intake area at or in the analyzer for receiving the cartridge with a data path for reading the storage unit;
an evaluation unit for evaluating the values measured by the analyzer; and
an output unit for printing an evaluation protocol.

8. The device according to claim 7, wherein the cartridge houses at least three sets of containers with at least five different measurement fluids.

9. The device according to claim 7, wherein the storage unit is realized as a data chip, which is attached to the cartridge.

10. The device according to claim 7, wherein the storage unit is realized as a data storage medium, which is contained in the package of the cartridge.

11. The device according to claim 7, wherein the evaluation unit and/or the output unit are parts of the analyzer.
